(19) United States
(12) Patent Application Publication
(10) Pub. No.: US 2016/0171596 A1
(43) Pub. Date: Jun. 16, 2016

(54) SELF-SERVICE PRESCRIPTION EYEWEAR KIOSK

(71) Applicant: ESSILOR INTERNATIONAL (COMPAGNIE GENERALE D'OPTIQUE), Charenton Le Pont (FR)

(72) Inventors: Jeff ANGERBAUER, Highland Village, TX (US); Benjamin R. BLACKBURN, Cheshire, CT (US)

(73) Assignee: Essilor International (Compagnie Generale D'Optique, Charenton Le Pont (FR)

(21) Appl. No.: 14/907,984

(22) PCT Filed: Jul. 25, 2014
(86) PCT No.: PCT/EP2014/066108

§ 371 Date: Jan. 27, 2016

Related U.S. Application Data
(60) Provisional application No. 61/859,020, filed on Jul. 26, 2013.

(51) Int. Cl. G06Q 30/06; A61B 3/11

(52) U.S. Cl. CPC G06Q 30/0643; G06Q 30/0631; A61B 3/111

(57) ABSTRACT

A self-service eyewear ordering kiosk is disclosed. Customers may order eyewear at the kiosk without requiring assistance from retail sales associates or from optical professionals. Sample eyewear frames are available at the kiosk for the customer to try on and to select a desired frame style. A user interface on the kiosk accepts customer eyewear data, such as a selected frame style code and an optical prescription. The kiosk also includes a camera that is used to capture images of the customer. The images are used to calculate at least one eyewear parameter on the customer, such as a pupillary distance. A lorgnette or other reference device is held to the customer's face to provide a reference for the eyewear parameter calculations.

### TECHNICAL FIELD

Embodiments of the invention are directed, in general, to providing an unaided, self-serve purchase solution for prescription eyewear, such as spectacle lenses, eyeglass frames, sunglasses, and reading glasses.

### BACKGROUND

Currently, customers seeking to obtain new eyeglasses are limited to making purchases at retail outlets. Such purchases usually require the assistance of an optician or a sales associate. Typically, the customer first obtains a prescription from an ophthalmologist and then visits an optician to choose a frame style for the eyeglasses. The customer may try several frames before selecting a desired frame. The optician then orders a pair of lenses corresponding to the prescription and adapted for the selected frames. Retail eyeglasses sales require trained opticians to take optical measurements, input the prescription, and order lenses from a laboratory.

The current process for purchasing eyeglasses limits the customer's choice as to location (i.e., at an optician's location) and timing (i.e., during the hours of the optician's retail operation). Additionally, in the current ordering process, customers may be subject to a high-pressure sales environment at the optician's retail outlet. These factors can lengthen the purchase cycle for prescription eyewear and may deter customers seeking to change frame styles or purchase extra eyeglasses. Consumers would benefit from a streamlined process that makes it easier and more economical to purchase prescriptive eyewear.

### BRIEF DESCRIPTION OF THE DRAWINGS

Having thus described the invention in general terms, reference will now be made to the accompanying drawings, wherein:

FIG. 1 illustrates a self-service prescription eyewear kiosk 100 according to one embodiment.

FIG. 2 is a block diagram of an example embodiment of a self-service prescription eyewear kiosk that communicates with a back end eyewear ordering system.

FIG. 3 is a flowchart illustrating a process for fulfilling customer orders for eyewear according to one embodiment.

FIGS. 4A and 4B are a flowchart illustrating a process for operating a self-service prescription eyewear kiosk according to one embodiment.

FIG. 5 illustrates a display for measuring pupillary distance (PD) according to one embodiment.

FIG. 6 is an illustration of a lorgnette used in one embodiment.

FIG. 7 illustrates a self-service prescription eyewear kiosk according to an alternative embodiment.

FIGS. 8A-8E are drawings illustrating additional details of a self-service prescription eyewear kiosk.

FIG. 9 illustrates a self-service prescription eyewear kiosk according to a further embodiment.

FIGS. 10A-10C is a flowchart illustrating a process for fulfilling customer orders for eyewear according to an alternative embodiment.

### DETAILED DESCRIPTION

The invention now will be described more fully hereinafter with reference to the accompanying drawings. This invention may, however, be embodied in many different forms and should not be construed as limited to the embodiments set forth herein. Rather, these embodiments are provided so that this disclosure will be thorough and complete, and will fully convey the scope of the invention to those skilled in the art. One skilled in the art may be able to use the various embodiments of the invention.

Example embodiments of a self-service prescription eyewear kiosk are disclosed. Customers seeking to purchase eyewear conforming to an optical prescription may use the kiosk instead of having to shop in retail environments. Customers may use the kiosk to obtain eyewear for a new prescription and/or to obtain secondary pairs of glasses for existing prescriptions. Customers may try on various frames styles and then directly order selected frame at the kiosk. The kiosk allows customers to evaluate lens upgrades and frame styles in a no-pressure sales environment. The kiosk also measures the customer's pupillary distance to ensure that the eyewear has a proper fit on the customer.

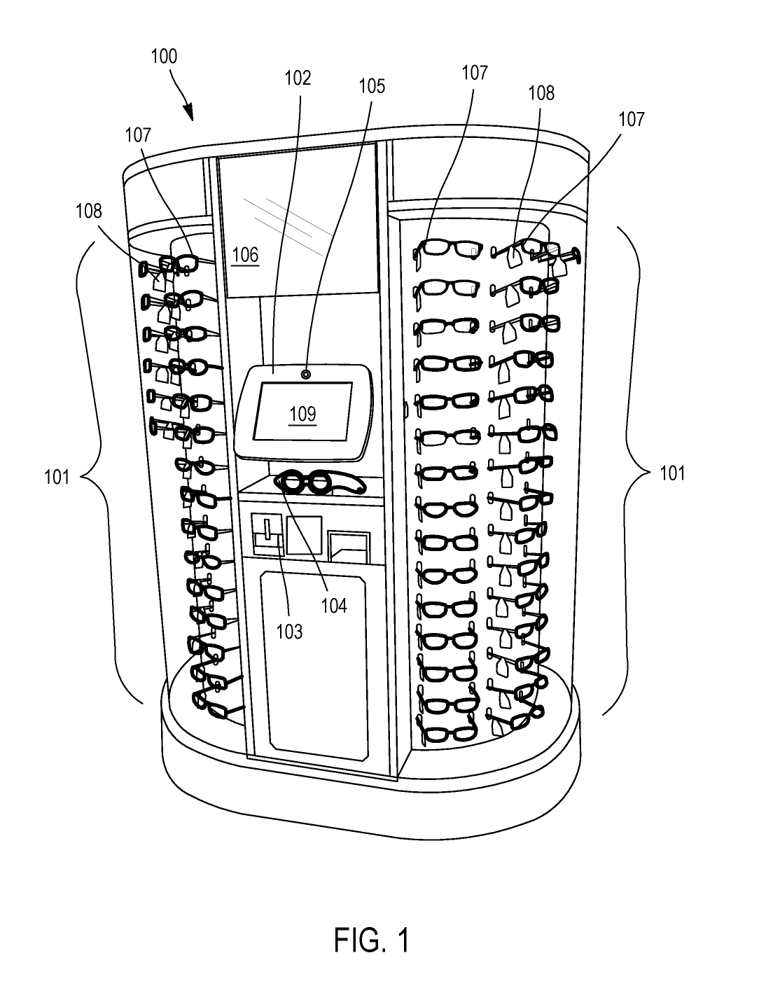

FIG. 1 illustrates a self-service prescription eyewear kiosk 100 according to one embodiment. Kiosk 100 comprises, for example, one or more frame display areas 101, user interface 102, payment interface 103, lorgnette 104, camera 105, and mirror 106.

Frame display areas 101 provide a selection of frames 107 for customers to try on at kiosk 100. Frames 107 may include a variety of styles and may be organized into categories, such as men's, women's, children's, and various designer styles. In one embodiment, sample frames 107 are for demonstration and style-selection purposes only and are not available for purchase by the customer. Frames 107 may have a tag 108 or other label, sticker, or other marking that indicates a unique frame code for a particular frame style. Customers may use mirror 106 when trying on and selecting a frame style.

When the customer has selected a desired frame style, he or she may purchase new eyeglasses via user interface 102. In one embodiment, user interface 102 has a touch screen 109 that allows the user to make selections and to provide inputs to an eyeglasses ordering application as described in detail below.

Lorgnette 104 and camera 105 may be used to measure the customer's pupillary distance, which is used to accurately center the lenses in the frame selected by the customer.

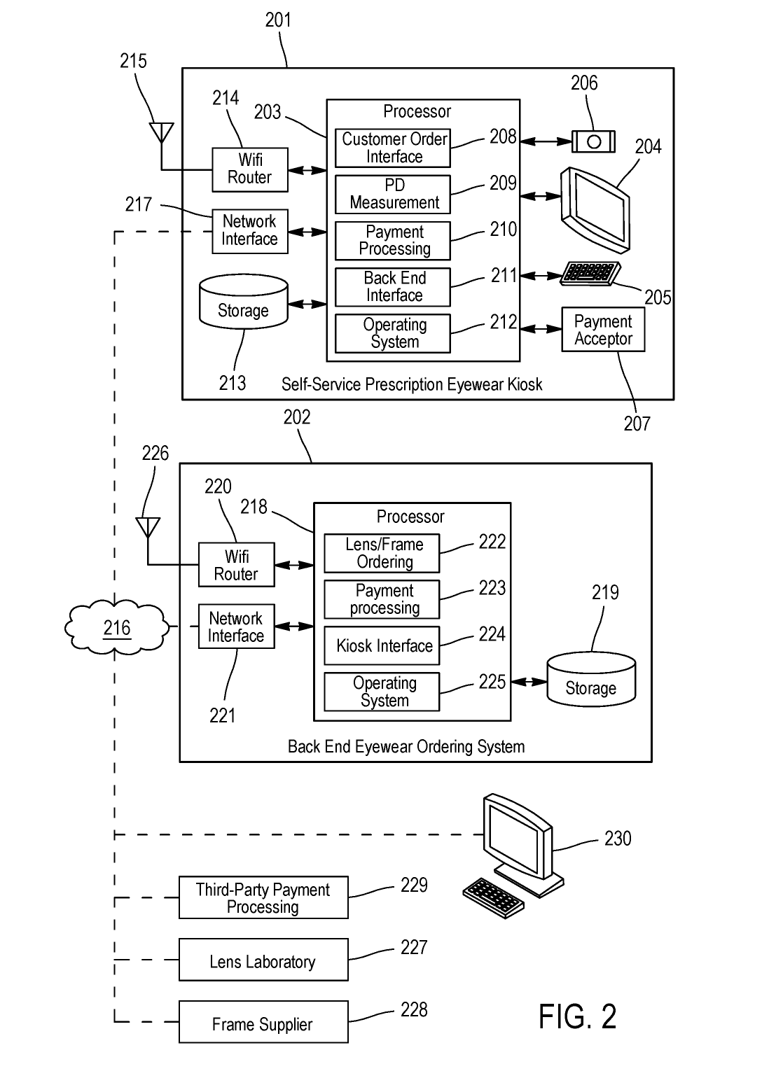

FIG. 2 is a block diagram of an example embodiment of a self-service prescription eyewear kiosk 201 that communicates with a back end eyewear ordering system 202. Kiosk 201 comprises a processor 203, user interface display 204, user interface keypad 205, camera 206, and payment acceptor 207. Processor 203 controls and coordinates the operation of kiosk 201 and its component using one or more software applications 208-212. Storage device 213 may be used to store software code and instructions for applications 208-212 and other data, such as customer account information and order information. Storage device 213 may be any appropriate memory device or computer storage media, such as, but not limited to, a hard disk drive, RAM, ROM, EEPROM, flash memory or other memory technology, CD-ROM, digital versatile disks (DVD) or other optical disk storage, magnetic cassettes, magnetic tape, magnetic disk storage or other magnetic storage devices. Kiosk 201 may communicate with back end eyewear ordering system 202 via a wireless or wired network connection. A WiFi router 214 or other wireless transceiver support wireless communications with back end system 202 either directly or through one or more intermediate access points or networks (not shown) using radio frequency (RF) signals transmitted and receive using antenna 215. In other embodiments, kiosk 201 communicates with back end system 202 via a public or private network(s) 216, such as the Internet, an intranet, or any other packet data network, which is accessed via a network interface 217.

Customer order interface application 208 provides outputs to display 204, such as instructions and other information for the customer. Customer order interface application 208 also receives input from the customer, such as via a touch-sensitive screen on display 204 or via keypad 205. In other embodiments, the customer may interact with kiosk 201 in any appropriate manner, such as using speech inputs, thumb scan, eye scan, and audio outputs, or any other similar input or output means. The user may input an optical prescription, selected frame style, account and mailing information, and any other appropriate or required information in response to visual or audio prompts generated by customer order interface 208.

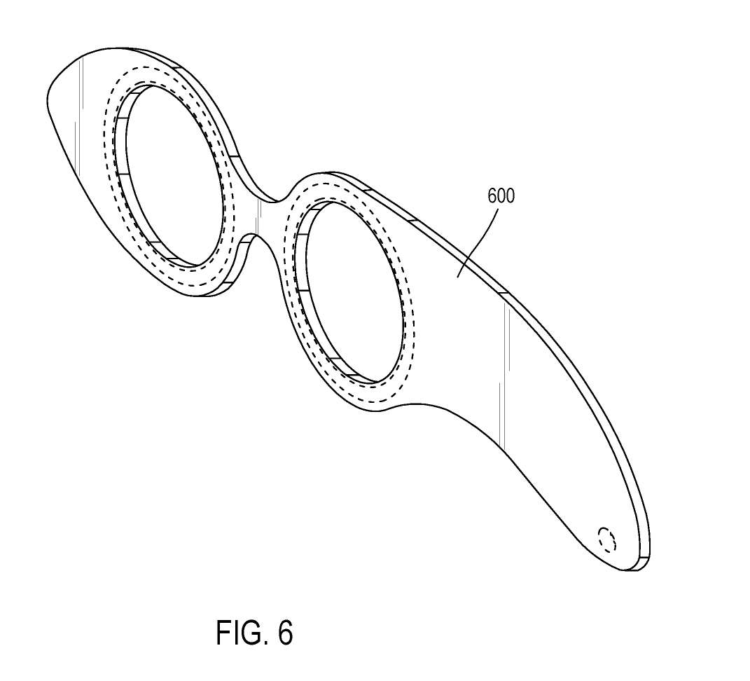

Pupillary distance (PD) measurement application 209 is used to calculate the customer's pupillary distance for use in manufacturing the selected eyewear. Pupillary Distance (PD) or interpupillary distance (IPD) is the distance (typically measured in millimeters) between the centers of the pupils in each eye. PD measurement application 209 may use images captured with camera 206 to calculate the pupillary distance. For example, as described in more detail below, the customer may hold lorgnette 104 (FIG. 1) up to the height of his or her eyes while camera 206 captures images of the customer's face. The lorgnette 104 is an apparatus having a solid unitary body with a first opening and a second opening spaced apart within the body to mirror the position of a user's eyes and a handle positioned on one side of the lorgnette. A user may grasp the lorgnette by using the handle, as illustrated in FIG. 6. PD measurement application 209 may then calculate the pupillary distance from the images using known dimensions or reference points on the lorgnette.

Payment processing application 210 is used to accept and process the customer's payment information. Payment processing application 210 may work with payment acceptor 207, which may accept credit/debit cards (e.g., magnetic card swipe), cash, tokens, coupons, gift cards, or other forms of payment.

Back end interface application 211 coordinates communication with back end eyewear ordering system 202. Back end interface application 211 exchanges customer account information, order information, payment data, software and system updates, and/or other data with back end eyewear ordering system 202.

Operating system 212 manages resources for processor 203 and provides services for applications 208-211 and other programs. Applications 208-211 may be components of operating system 212 or may be separate software programs running on processor 203.

In one embodiment, some components of self-service prescription eyewear kiosk 201 may correspond to a portable computing device, such as a tablet computer, smartphone, PDA, laptop computer, notebook computer, or the like. For example, processor 203, storage 213, user interface display 204, and camera 206 may be elements of a tablet computer (or other portable computing device) that is mounted on a kiosk 201. Similarly, user interface 102, touch screen 109, and camera 105 on kiosk 100 (FIG. 1) may also be elements of a tablet computer or portable computing device.

Back end eyewear ordering system 202 may be connected to one or more kiosks, such as a plurality of self-service kiosks 201 deployed in different retail locations. System 202 provides support for completing customer orders, processing payment transactions, ordering lens and frames, etc. Back end eyewear ordering system 202 may be a server or other computer, for example, comprising processor 218, storage device 219, WiFi router 220, and/or network interface 221.

Processor 218 controls and coordinates the operation of system 202 and its component using one or more software applications 222-225. Storage device 219 may be used to store software code and instructions for applications 222-225 and other data, such as customer account information and order information Storage device 219 may be any appropriate memory device or computer storage media, such as, but not limited to, a hard disk drive, RAM, ROM, EEPROM, flash memory or other memory technology, CD-ROM, digital versatile disks (DVD) or other optical disk storage, magnetic cassettes, magnetic tape, magnetic disk storage or other magnetic storage devices. System 202 may communicate with kiosk 201 via a wireless or wired network connection. WiFi router 220 or another wireless transceiver support wireless communications with kiosk 201 either directly or through one or more intermediate access points or networks (not shown) using RF signals transmitted and received using antenna 226. In other embodiments, back end system 202 communicates with kiosk 201 via a public or private network(s) 216, which is accessed via a network interface 221.

Lens and frame ordering application 222 provides support for fulfilling customer orders by ordering or scheduling production of lenses and frames from a lens laboratory 227 and/or a frame supplier 228. System 202 and/or kiosk 201 may communicate with lens laboratory 227 and/or frame supplier 228 via network 216, for example.

Payment processing application 223 is used to process customers' payment information and to interface with third-party payment processor 229. Payment processing application 223 may receive payment information from payment processing application 210 on kiosk 201. Payment processing application 223 may communicate with third-party payment processor 229 via network 216, for example.

Kiosk interface application 224 provides communication between system 202 and a plurality of kiosks 201 to support, for example, receiving customer orders, monitoring the status of kiosk 201, providing software updates and other data to kiosk 201, and/or any other information.

Operating system 225 manages resources for processor 218 and provides services for applications 222-224 and other programs. Applications 222-224 may be components of operating system 225 or may be separate software programs running on processor 218.

In other embodiments, a customer may also order prescription eyewear using a remote terminal 230, which may be, for example, a desktop or personal computer, laptop computer, tablet computer, smartphone, mobile computing device, personal digital assistance (PDA), or other processor-based device that is capable of connecting to back end eyewear ordering system 202 and/or kiosk 201. The customer may initiate and save a new order at terminal 230, or may complete or revise an existing order started on kiosk 201. For example, the customer may start an order at a kiosk, and then finish the order at home using any type of remote terminal, such as a laptop, notebook, tablet, mobile phone, PDA, desktop computer, or the same or a different kiosk at a later time.

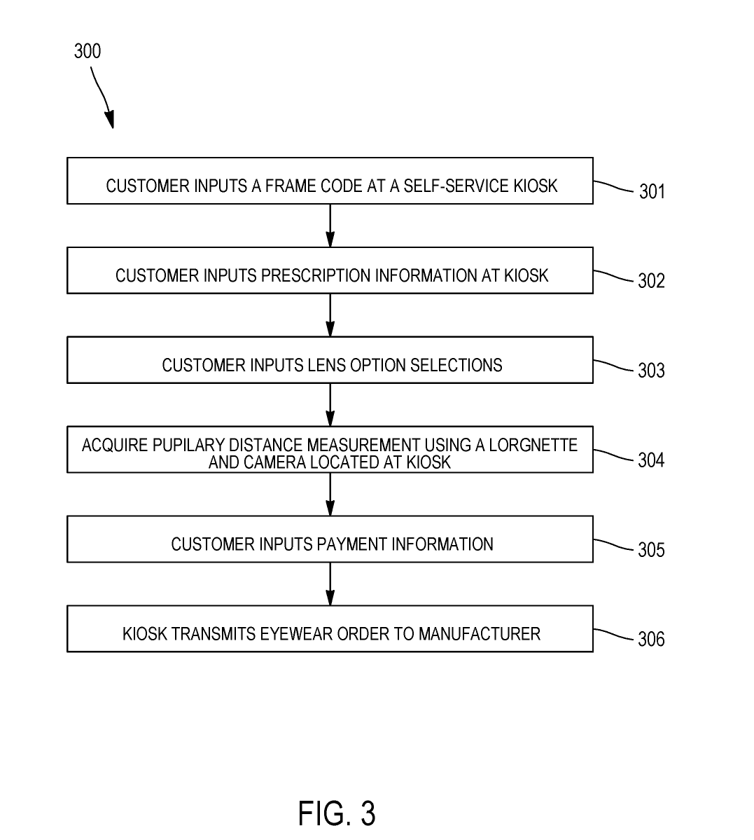

FIG. 3 is a flowchart 300 illustrating a process for fulfilling customer orders for eyewear according to one embodiment. A customer visits a self-service eyewear kiosk, such as kiosk 100 or 201, for example. The customer may try on and evaluate various frame styles offered at the kiosk. When the customer selects a desired frame style, they may log on or otherwise initiate an eyewear ordering application on the kiosk. In step 301, the customer enters a code for the selected frames. The code may be uniquely assigned to a particular frame design and size and/or may indicate sizes for elements of the selected frame, such as the eye size (horizontal width the frame's lenses), bridge size (distance between the two lenses), temple size (length of the temple or ear piece), vertical size (vertical height of the lens), colors or patterns on the frame, frame manufacturer, or other features of the selected frames. In one embodiment, a three character alphanumeric code is used to identify frame styles.

Although example embodiments described herein refer to prescription eyewear, it will be understood that the self-service eyewear kiosk may be used to purchase any type of eyewear. For example, the customer may purchase prescription and non-prescription eyeglasses, reading glasses, sunglasses, protective eyewear, safety goggles, and athletic eyewear.

Once the customer has identified his or her frame selection, the customer inputs his or her optical prescription at the kiosk at step 302. The customer may use a touchscreen interface on the kiosk to enter his or her prescription information. The prescription input interface may provide, for example, fields for entry of distance vision and near vision information, such as a spherical correction, cylindrical correction, axis, prism, and base, as are known to those of skill in the area of ophthalmology and eyewear manufacturing. In step 303, the customer may also select ophthalmic lenses and enter lens options.

In step 304, a pupillary distance measurement is acquired at the kiosk using a camera and lorgnette. The customer holds the lorgnette up to his or her eyes, and the camera then captures one or more images of the customer's face with the lorgnette in place. A pupillary distance measurement application on the kiosk or on a back-end server processes the images and calculates the pupillary distance based on known parameters of the lorgnette.

In step 305, the customer inputs payment information at the kiosk to complete the purchase of his or her eyewear, which includes the selected frame and lenses corresponding to the input prescription. The customer may use a credit/debit card, gift card, or any other form of currency for payment, such as by swiping the card in a magnetic card reader. The kiosk provides the payment information to a back end server and/or a third-party payment service to complete the payment transaction.

In step 306, the kiosk transmits the eyewear order to a manufacturer. For example, the kiosk may send the optical prescription and frame parameters to a lens laboratory. The kiosk may send the order directly to the manufacturer, or the order may be routed through a central server.

In other embodiments, the customers may order more specialized lenses for his or her eyewear using the kiosk. For example, customers may order single vision, bifocal, trifocal, progressive, and prism lenses. Customers may also select other non-prescription lens options, such as lens material (e.g., glass, polycarbonate, plastic, etc.), coating (e.g., antireflective, no glare, UV protection, scratch resistance, anti-static, smudge resistance, water repellence, etc.), tinting, polarization, and/or photochromic (e.g., adapt to varying light conditions). The customer may also use self-service prescription eyewear kiosk to order contact lenses.

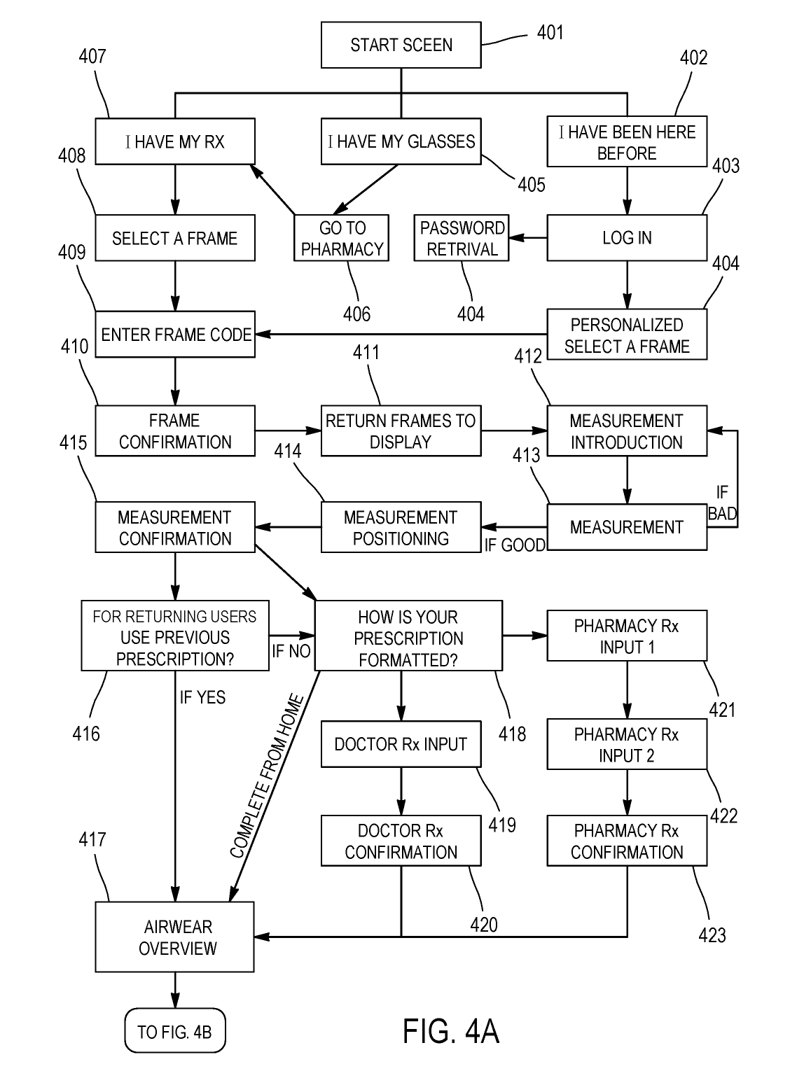

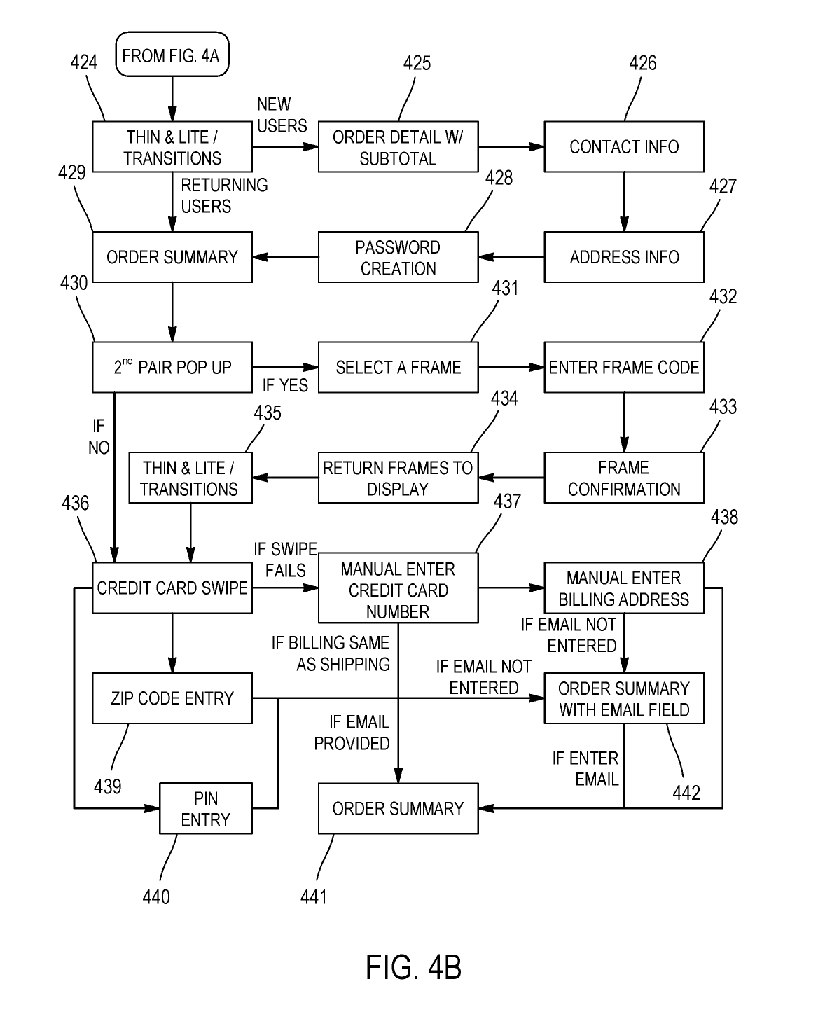

FIGS. 4A and 4B are a flowchart illustrating a process for operating a self-service prescription eyewear kiosk according to one embodiment. The customer is presented with a start screen in step 401, which allows the customer to indicate whether they have an optical prescription, a current pair of eyeglasses, an existing account, or other options. In step 402, the customer has indicated that they have used the kiosk previously. This allows the customer to access an existing account, update existing or saved orders, and the like. In step 403, the kiosk displays a log-in screen on which that the customer enters identification and/or account information. In step 404, the kiosk allows the customer to retrieve a password, if needed. Once the customer is to the kiosk provides the customer with a personalized process for selecting frames in step 404.

If the customer indicates that he or she has a current pair of eyeglasses, but not the details of his or her optical prescription, then the process moves to step 405 from start screen 401. The kiosk then indicates in step 406 that the customer should go to the pharmacy (e.g., if appropriate for the kiosk location) where the customer can obtain an optical prescription for the existing eyeglasses. For example, the pharmacy may use a device to scan the customer's current eyewear in order to generate a prescription. In other embodiments, if the customer does not have a prescription, the kiosk may direct the customer to another location (e.g., an autorefraction device or station or an optometrist or ophthalmologist office) where the customer can obtain an optical prescription.

The process moves to step 407 if the customer indicates at start screen 401 that he or she has an optical prescription or after a prescription has been obtained in step 406. In step 408, the customer selects a frame. The customer may select the frame from samples located at the kiosk, from a catalog or other listing, or from any other source. In step 409, the customer enters a frame code to identify the selected frame style. The customer's frame selection is confirmed in step 410. The kiosk may remind the customer to return the sample frames to the display rack in step 411.

The kiosk begins a measurement sub-process in step 412. The measurement sub-process may include entering the customer's optical prescription and measuring pupillary distance for the customer. In step 413, the pupillary distance may be measured using a reference device, such as a lorgnette, attached to the kiosk. If the customer's pupillary distance measurement fails in step 413, the measurement sub-process returns to step 412 and starts again. If the pupillary measurement is good in step 413, the process moves to measurement positioning in step 414 and then measurement confirmation in step 415.

In step 416, the process evaluates whether this is a returning customer and offers the option to use an existing optical prescription. If the customer does want to use the existing prescription, then the process moves to step 417 where the kiosk provides information about various lens options to the customer, such as Airwear® lenses manufactured by Essilor International Corporation.

If the customer does not want to use an existing prescription in step 416, then the kiosk requests information about how the prescription is formatted in step 418. If the prescription is from an ophthalmologist or doctor, then the process moves to step 419 where the customer enters the prescription. The prescription is then confirmed in step 420. If the customer has obtained a prescription from the pharmacy (or other source referred to in step 406), then the customer enters the prescription at steps 421 and 422 following kiosk prompts that are appropriate for the pharmacy prescription format. In step 423, the process confirms the pharmacy prescription.

Once the customer has entered and confirmed either an ophthalmologist or pharmacy prescription, the process moves to step 417 where the kiosk provides information about various lens options to the customer. The customer may select lens options, such as lightweight (e.g., thin & lite) or photochromatic (e.g., Transition®) lenses, in step 424 (FIG. 4B).

After the customer has entered a prescription and selected lens options, the process provides order information. New customers may be provided order details with a subtotal in step 425. The new customer may then provide contact information in step 426, address information in step 427, and create an account password in step 428. The customer may later access the account and review orders from this or another kiosk. The customer may also use the password from a remote terminal, for example, an Internet webpage or using a mobile application. The customer is then shown an order summary in step 429. Returning customers, who do not need to create an account or update contact or address information, may skip steps 425-428 and are provided an order summary in step 429.

The customer is offered the option to select a second pair of eyeglasses in step 430. If the customer elects to order a second pair, then he or she selects a second frame from the display in step 431 and enters the code for the selected frames in step 432. The frame selection is confirmed in step 433 and the kiosk reminds the customer to return the sample frames to the display rack in step 434. The customer is then provided with lens options for the second pair in step 435. Once the customer has completed entering the frame and lens selections in steps 431-435, the process moves to a payment sub-process starting at step 436. Customers may also have the option (not shown) of ordering additional pairs of eyeglasses by repeating steps 431-435 as needed. Customers who do not order a second pair of eyeglasses skip steps 431-435 and move directly to step 436 after step 430.

In step 436, the customer swipes a credit or debit card, or otherwise initiates payment. In other embodiments, the customer may pay using cash, tokens, coupons, gift cards, or other exchange of value. If a credit card swipe fails in step 436, the customer may manually enter the credit card number in step 437. The customer then provides a billing address in step 438. If the credit card swipe works in step 436, then the process moves to either step 439 or 440 where the customer is prompted to enter a zip code or PIN as appropriate for the card used.

After payment information has been received, the process provides the customer with an order summary in step 441. If the customer has not provided an email address, then the order summary may include an email address entry field in step 442. Once the customer has provided all required information and acknowledges the order, the kiosk will send the order information to a back end system, central server, or other location to initiate manufacture of the eyewear.

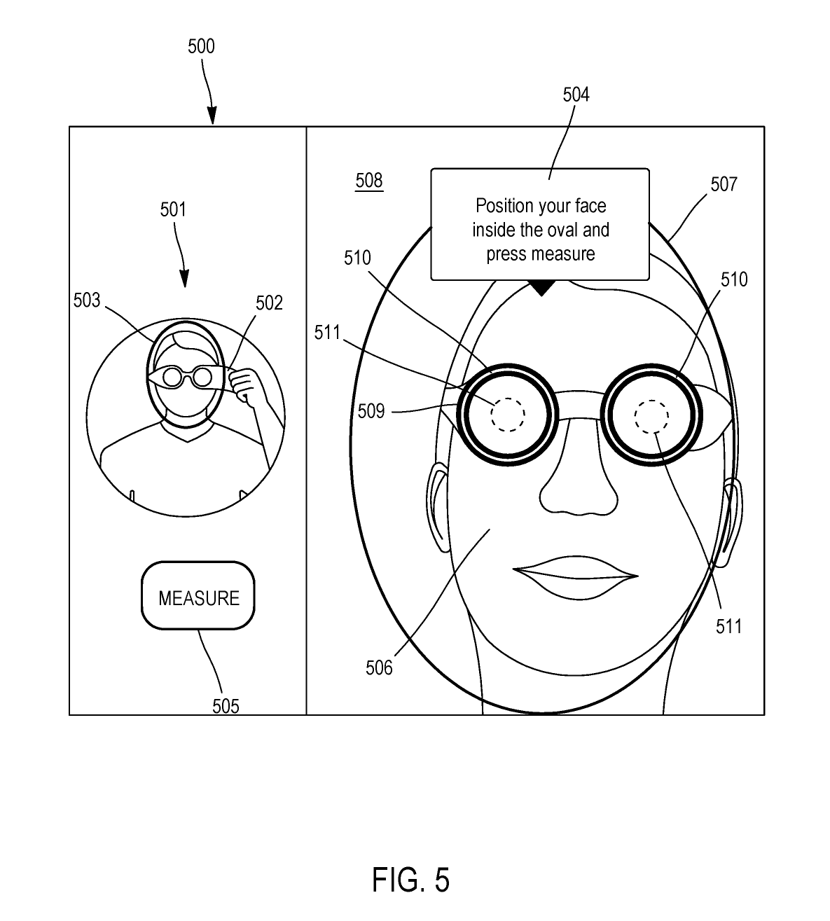

FIG. 5 illustrates a display 500 for measuring pupillary distance according to one embodiment. A lorgnette, user interface display, and camera (e.g., lorgnette 104, camera 105, and display 109 in FIG. 1) may be used to measure pupillary distance. The kiosk may provide visual and/or written instructions that direct the customer through the steps required for the pupillary measurement. Visual instructions 501 direct the customer to hold lorgnette 502 up to the height of his or her eyes such that one eye of the customer is substantially visibly positioned within the first opening of the lorgnette and the other eye of the customer is substantially visibly positioned within the second opening of the lorgnette. Visual instructions 501 further direct the customer to position his or her face within a frame of reference, such as, but not limited to, oval 503 on the display. Additionally or alternatively, written instructions 504 direct the user to position his or her face within the oval and to press a “measure” button 505.

The customer positions his or her face 506 within oval 507 on the display 508 and holds lorgnette 509 up to the height of his or her eyes. The customer then presses the measure button 505 or provides any other appropriate input. The kiosk then captures one or more images of the customer's face 506 with the lorgnette 509 in place. An application on the kiosk then measures the pupillary distance. Rings 510 on lorgnette 509 may be used as reference points or as a known dimension. The pupillary distance application identifies the customer's eyes, irises, or pupils and positions rings 511. In one embodiment, the customer's pupils are located by identifying corneal reflections in the captured images. The relative sizes and locations of the lorgnette rings 510 and eye rings 511 are used to calculate the pupillary distance. In one embodiment, the customer positions his or her head in the kiosk display to align white lorgnette rings 510 with green circles on the display to ensure proper positioning and accuracy of the measurements. Once the circles are aligned, the customer's image is captured and processed to identify the customer's eyes, irises, and pupils for the PD measurement. In other embodiments, instead of using rings 511, the location of the customer's pupils may be identified by points designating the center of the pupils.

In one embodiment, the camera and lorgnette are used to focus on the customer's eyes. Multiple images of the customer's face may be taken and an image with high contrast is selected for measurements. An image sensor is used to find the image with the highest PD. Instead of requiring the customer to press “measure” or otherwise initiate image capture, the camera may use facial recognition software to identify when a face and lorgnette are in view and then automatically capture images of the customer.

FIG. 6 is an illustration of a lorgnette 600 used in one embodiment. Lorgnette 600 may be used to determine the PD measurement for a customer as described above.

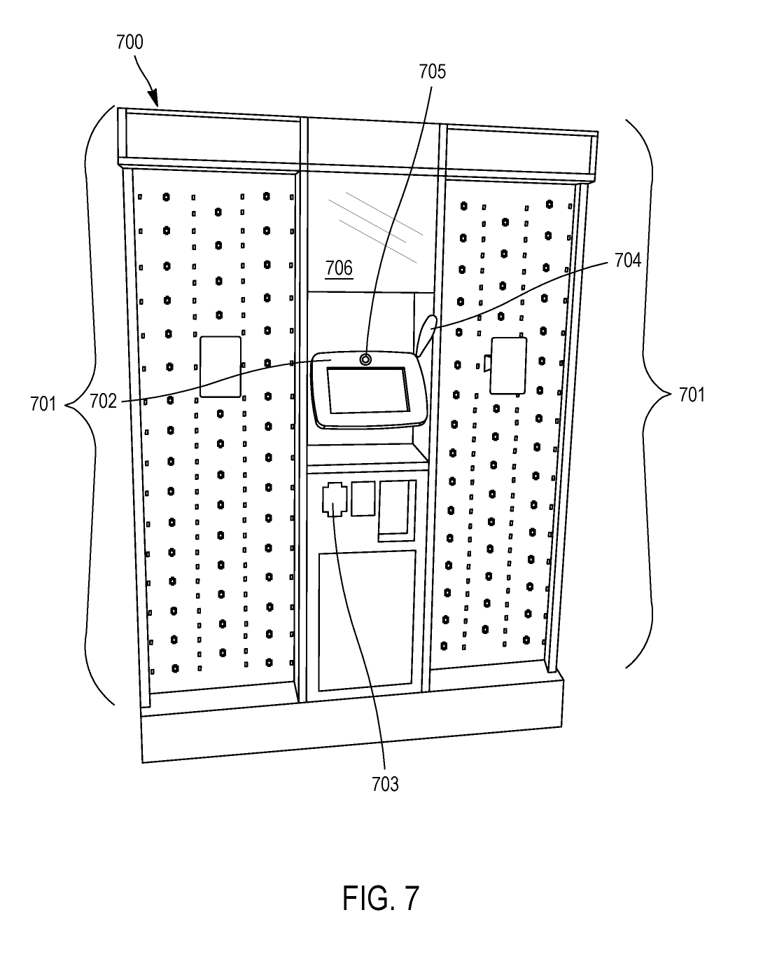

FIG. 7 illustrates a self-service prescription eyewear kiosk 700 according to an alternative embodiment. Kiosk 700 comprises, for example, one or more frame display areas 701, user interface 702, payment interface 703, lorgnette 704, camera 705, and mirror 706.

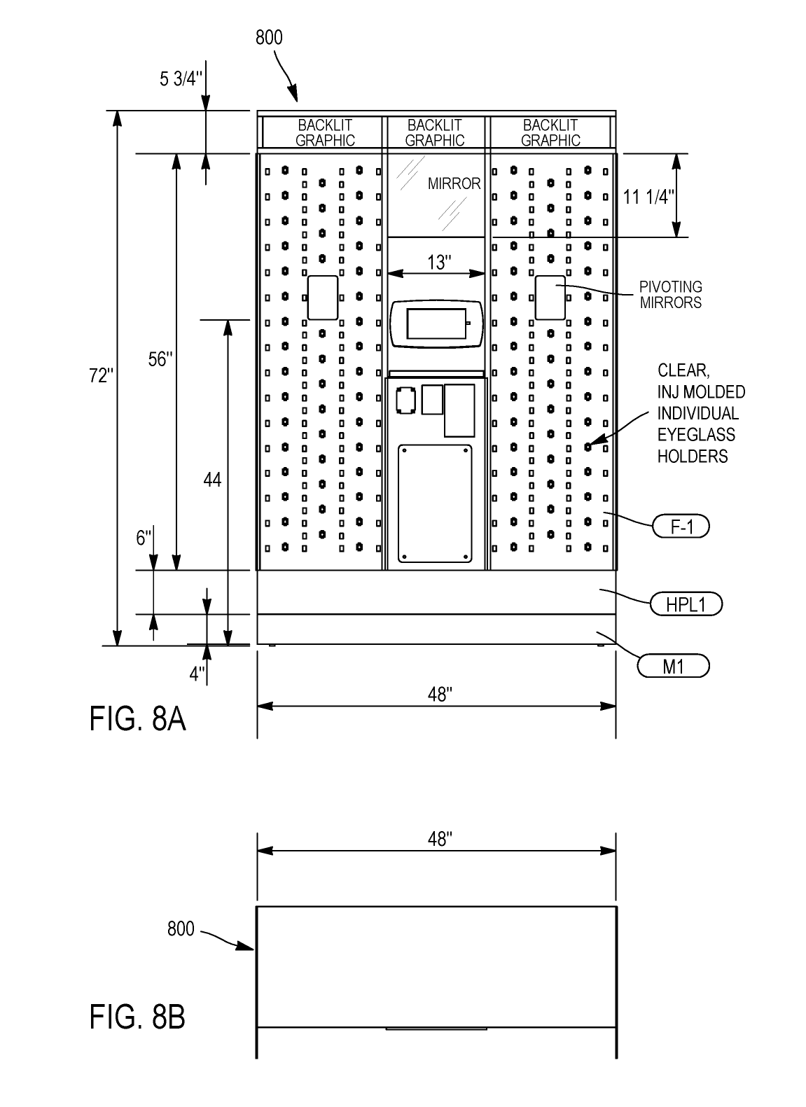

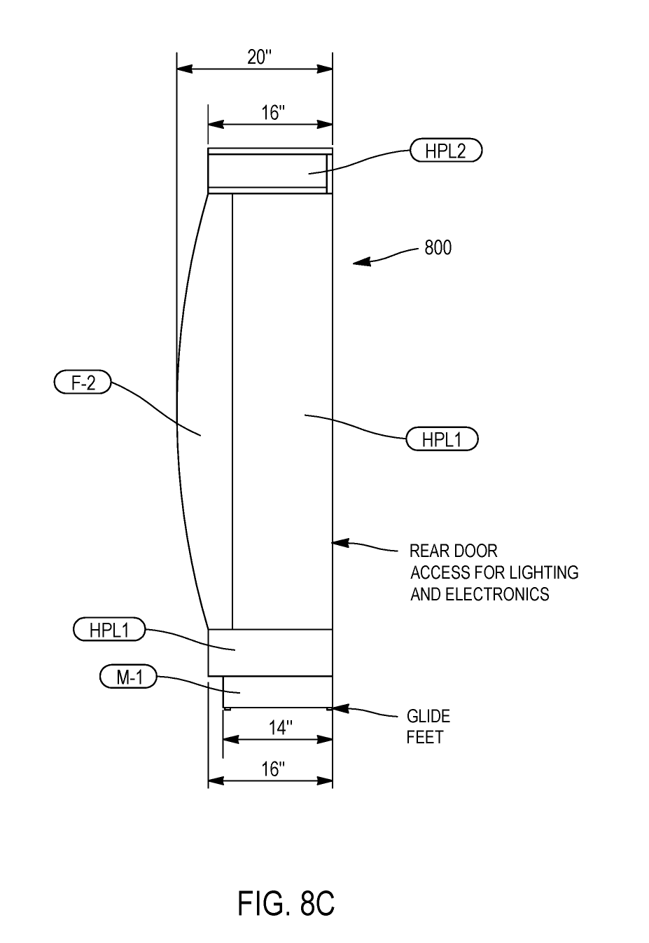

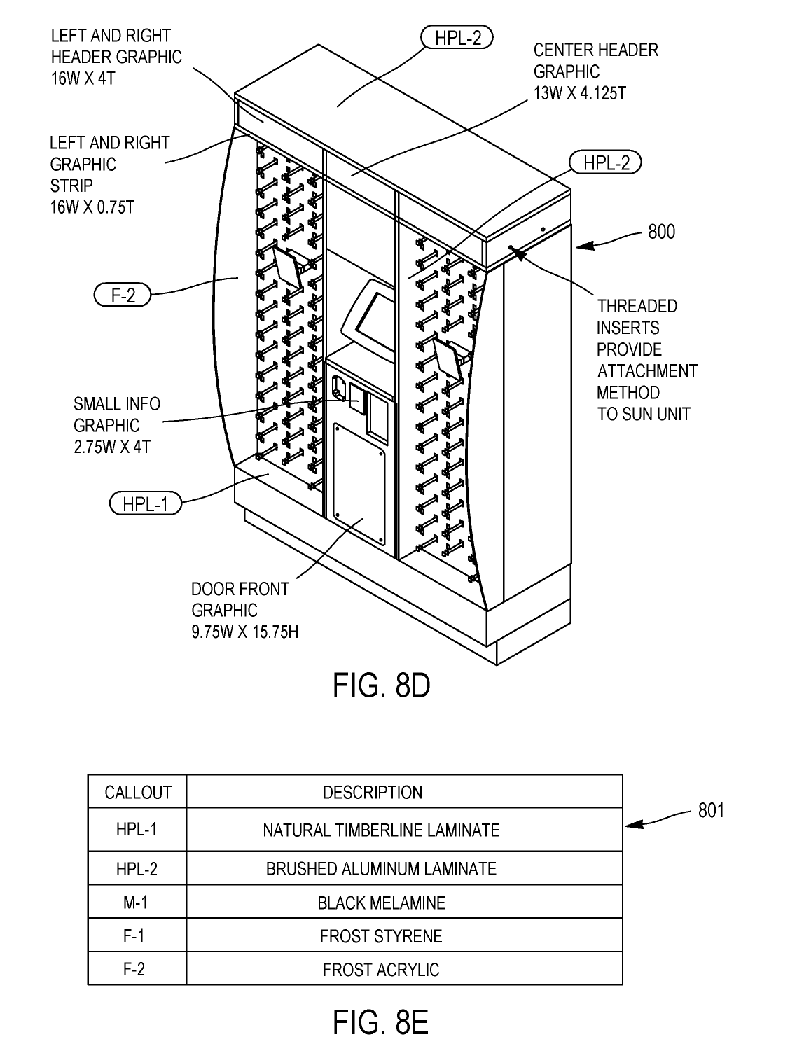

FIGS. 8A-8E are drawings illustrating additional details of a self-service prescription eyewear kiosk 800, which may correspond to kiosk embodiment 700 (FIG. 7), for example. FIG. 8A is a front view of kiosk 800. FIG. 8B is a top view of kiosk 800. FIG. 8C is a side view of kiosk 800. FIG. 8D is an isometric view of kiosk 800. FIG. 8A is table 801 of materials used in an example embodiment of kiosk 800.

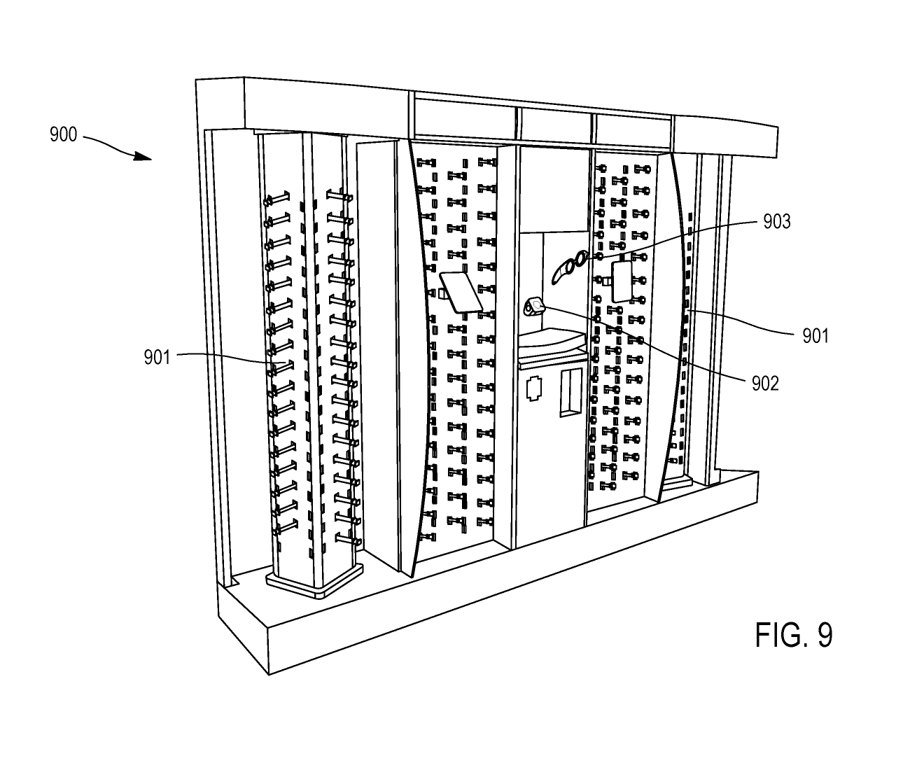

FIG. 9 illustrates a self-service prescription eyewear kiosk 900 according to a further embodiment. Kiosk 900 comprises, for example, kiosk 701 plus additional frame display areas 901. Kiosk 900 further comprises a portable computing device docking port 902. Docking port 902 may be used with any appropriate portable computing device, such as a smartphone (e.g., iPhone or mobile phone running an Android or Windows Phone operating system), tablet computer (e.g., iPad, Microsoft Surface, or Android-based tablet), notebook computer, laptop computer, or the like. Docking port 902 may provide one or more connectors, such as a USB connector, Apple Lightning connector, Apple 30-pin connector, or any other well-known or proprietary connector, that can be used to connect the portable computing device to kiosk 900. Docking port 902 may also be adapted to hold the portable computing device, such as a rack or frame configured to hold a smartphone, tablet, or other device.

In one embodiment, the processor for kiosk 900 is the customer's own portable computing device, such as a private portable tablet, that can download and run an eyewear measurement application. This would allow an eyewear kiosk to be provided without its own processor device. Instead, the customer may download the eyewear measurement application, such as from an online application store (e.g., an “App Store”) or from an eyewear manufacturer's website. Alternatively, the kiosk 900 may provide such an application for download locally. For example, the customer may scan a bar code or Quick Response (QR) code on the kiosk to access a site to download the eyewear measurement application. The user's portable computing device may be used to acquire images of the user's face as described herein if the device includes a camera. For example, the customer's smartphone, tablet computer, or portable computing device may correspond to some or all of the components illustrated as kiosk components 201 (FIG. 2). Other embodiments may include a separate camera located on kiosk 900. A lorgnette 903 may be located at and/or attached to the kiosk 900 for use by any customer who has their own portable computing device.

The incorporation of the user's own portable computing device allows the user to initiate an eyewear order at kiosk 900 and then complete the order at any location. For example, the user may use lorgnette 903 at kiosk 900 to capture images for pupillary distance measurements. The user may then finish his or her order at some other location and/or at some other time once the required images have been captured.

In other embodiments, once a customer has selected a frame style from kiosk 900 or kiosk 100 (FIG. 1), he or she may then complete the measurements on his or her own smartphone, tablet computer, or portable computing device at home or some other location away from the kiosk. Once the customer has downloaded the eyewear measurement application on their device, they may complete the PD measurements using their device in any location. The eyewear measurement application will prompt the customer to complete the measurements as illustrated in FIG. 5 using the display and camera on the customer's device. The customer may use a lorgnette that they obtained at the kiosk (e.g., a disposable or give-away lorgnette) for the measurements on their own device.

The user may send his or her order to a central system, such as back end eyewear ordering system 202 (FIG. 2), which verifies and completes the order. The central system may evaluate the feasibility of the order, such as checking available frame and lens stock, determining if pupillary distance or other eyewear parameters are compatible with selected frames, identifying potential errors or problems with the user's prescription, etc. The central system may send an acknowledgement to confirm the user's order once it has confirmed the order is feasible. The central system may provide additional information such as shipping and delivery dates, shipping tracking codes, order confirmation numbers, etc.

In other embodiments, the process for ordering eyewear and/or measuring eyewear parameters may run on the central server instead of running as an application on the portable computing device. The central server may provide a user interface via a web browser, for example, to collect information, images, and other data for processing on the central server.

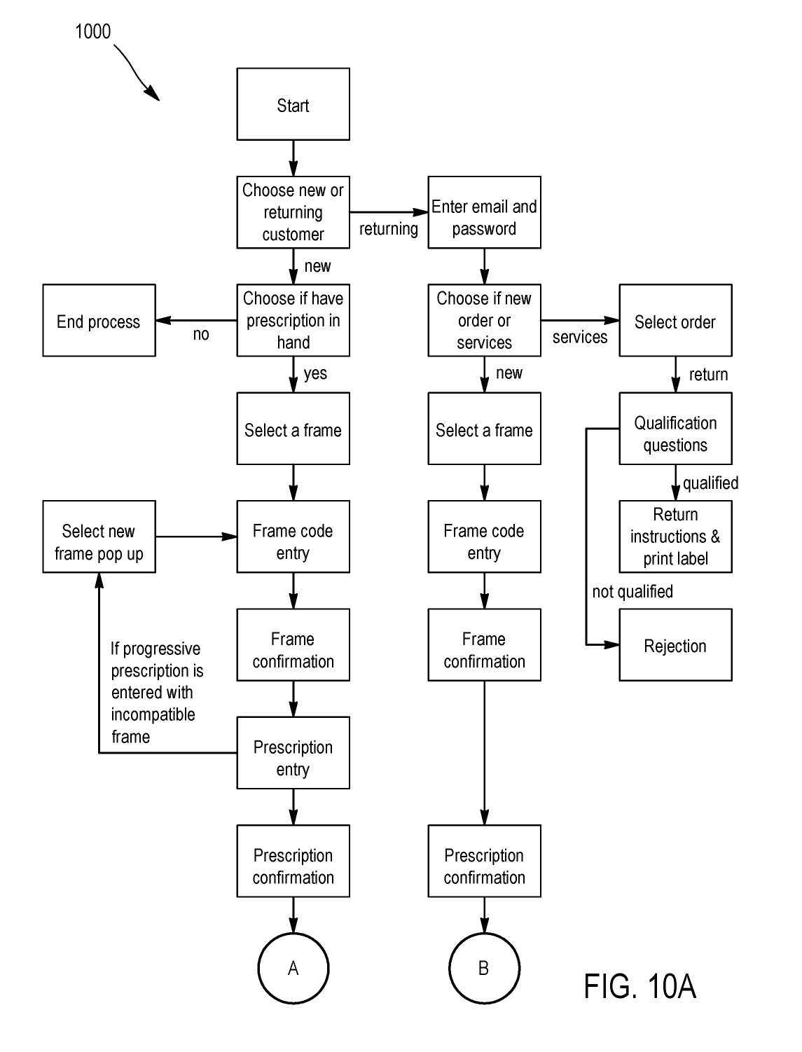

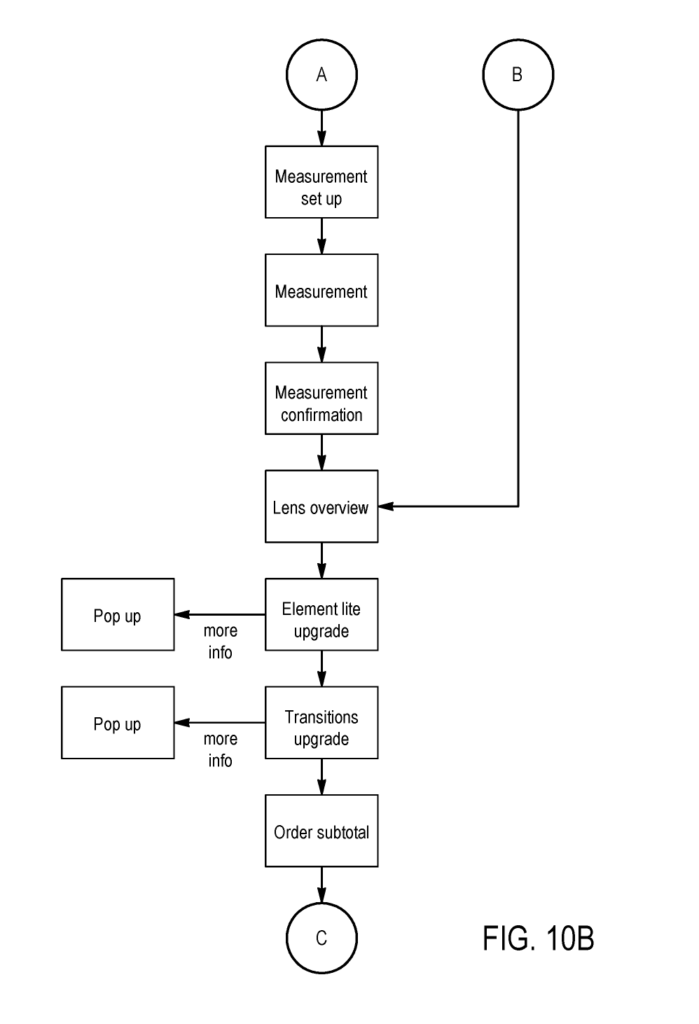

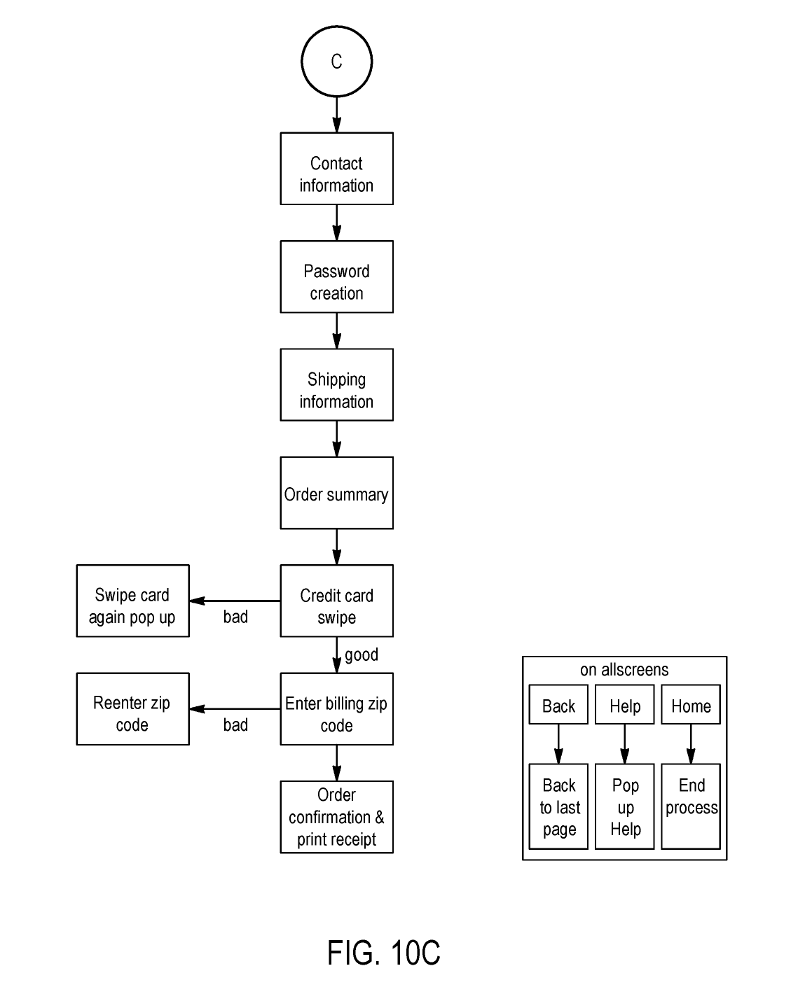

FIGS. 10A-10C is a flowchart 1000 illustrating a process for fulfilling customer orders for eyewear according to an alternative embodiment.

Many modifications and other embodiments of the invention will come to mind to one skilled in the art to which this invention pertains having the benefit of the teachings presented in the foregoing descriptions, and the associated drawings. Therefore, it is to be understood that the invention is not to be limited to the specific embodiments disclosed. Although specific terms are employed herein, they are used in a generic and descriptive sense only and not for purposes of limitation.

What is claimed is:

1. A self-service kiosk for ordering prescription eyewear, comprising:
a user interface configured to receive prescription eyewear order information directly from a customer, the order information comprising a frame style and an optical prescription;
a camera configured to capture images of a customer inputting order information at the user interface;
a processor configured to receive image data from the camera; and
computer-readable storage media having stored thereon computer-executable instructions that, when executed by the processor, causes the processor to perform a method for calculating eyewear measurements for the customer, the method comprising:
presenting an image of the customer's face on the display;
capturing images using the camera when the customer's face is located in a desired position relative to a reference; and
calculating the eyewear measurements using one or more selected images.

2. The self-service kiosk of claim 1, wherein the eyewear measurements are a pupillary distance calculated using one or more of the images.

3. The self-service kiosk of claim 1, wherein the reference is a lorgnette that is positioned in front of the customer's eyes.

4. The self-service kiosk of claim 1, the processor further operating to:
identify a customer eye position in a selected image using a reflection on a cornea of the customer's eye.

5. The self-service kiosk of claim 1, wherein the reference is a shape presented on the user interface display, the shape designating an area wherein the customer's face should be positioned.

6. A method for providing eyewear, comprising:
receiving an eyewear frame identifier from a user;
capturing one or more images of the user, the images comprising the user's face in proximity to a reference device or lorgnette;
calculating at least one eyewear parameter using information in a selected image; and
generating an order for the eyewear based upon the frame identifier and the eyewear parameter.

7. The method of claim 6, wherein the one or more images are captured using a camera on a portable computing device.

8. The method of claim 6, further comprising:
receiving optical prescription data from the user, and
wherein the order for the eyewear is further based upon the optical prescription data.

9. The method of claim 6, further comprising:
identifying user eye positions using corneal reflections in the selected image; and
calculating a pupillary distance for the user based upon the user eye positions.

10. The method of claim 6, wherein the eyewear frame identifier is received from the user and the images of the user are captured at a self-service kiosk; and
the method further comprising:
receiving a plurality of eyewear orders at a central server from one or more kiosks.

11. The method of claim 10, further comprising:
forwarding eyewear order data from the central server to a lens laboratory.

12. A system for receiving eyewear order information from customers, comprising:
a lorgnette reference device that is configured for use in measuring at least one eyewear parameter for a customer;
a camera configured to capture images of the customer's face with the lorgnette reference device positioned near the customer's eyes; and
a processor configured to calculate the at least one customer eyewear parameter from a captured image.

13. The system of claim 12, wherein the camera and processor are components of a portable computer device.

14. The system of claim 13, wherein the portable computer device comprises:
a user interface for receiving eyewear frame selection data from the customer; and
a wireless network interface configured to communicate eyewear order information to a remote device.

15. The system of claim 13, further comprising:
a self-service kiosk configured to hold the table computer for use by the customer, the self-service kiosk comprising a docking port configured to attach to a customer's portable computer device.
